(19) United States
(12) Patent Application Publication
(10) Pub. No.: US 2025/0186168 A1
(43) Pub. Date: Jun. 12, 2025

(54) INSTRUMENT HEAD WITH LEVER MECHANISM FOR CLAMPING A TREATMENT TOOL

(71) Applicant: KAVO DENTAL GMBH, Biberach an der Riss (DE)

(72) Inventors: Jürgen BUTSCHER, Leutkirch (DE); Thomas RÖSCH, Warthausen (DE)

(21) Appl. No.: 18/845,330

(22) PCT Filed: Feb. 22, 2023
(86) PCT No.: PCT/EP23/54416

§ 371 Date: Sep. 9, 2024

(30) Foreign Application Priority Data
Mar. 10, 2022 (DE) 10 2022 105 626.0

(51) Int. Cl. A61C 1/14; A61C 1/16

(52) U.S. Cl. CPC A61C 1/14; A61C 1/16

(57) ABSTRACT

A medical, in particular dental hand-held instrument (1) has, on its headpiece (20), a cover (30) which has a lever mechanism (304) for releasing a holding mechanism arranged in the interior of the headpiece (20). The lever mechanism (304) has, in this case, a first lever (320) and a second lever (330), wherein, upon actuation (B) of the first lever (320), the second lever (330) interacts with the holding mechanism and releases the latter such that a treatment tool (7) can be coupled to or uncoupled from the headpiece (20) of the hand-held instrument (1).

### BACKGROUND OF THE INVENTION

The invention relates to a medical, in particular dental, hand-held instrument having an instrument head and a medical treatment tool received in the instrument head, wherein the treatment tool is releasably coupled to the instrument head via a holding mechanism.

Hand-held instruments of this kind usually consist substantially of a grip sleeve and an instrument head, also referred to as a headpiece, which, for easy and precise operation of the hand-held instrument, is arranged at a front end of the grip sleeve. FIG. 6 shows such a conventional headpiece 20, which is arranged at one end of a grip sleeve 10 of a medical hand-held instrument 1. The headpiece 20 has, in that case, a tool receiving opening 240 such that, depending on the application, different medical or dental treatment tools 7 can be inserted into the headpiece 20 and be coupled to the hand-held instrument 1. To that end, a holding means 230 for rotatably securing the treatment tool 7 in question, for example a dental drill, is present in the interior of the headpiece 20. Also coupled to the holding means 230 is a release element 210 which, upon corresponding actuation, releases the securing of the treatment tool 7 in the holding means 230. During operation, the treatment tool 7 and the components that are coupled to the treatment tool 7, such as the release element 210 and holding means 230, rotate at up to 450 000 revolutions per minute.

Hitherto, to secure the treatment tool 7, use has usually been made of a collet 230, which is actuated by means of the wedge-shaped release element 210 by pressing a push button 1000 such that the securing of the treatment tool 7 is released. To this end, the push button 1000 usually has a pressure element 1001 and a compression spring 1040, which pushes the push button 1000 away counter to the actuation direction of the push button 1000. Upon actuation of the push button 1000, the compression spring 1040 is compressed, wherein the pressure element 1001 then contacts the release element 210 and displaces the latter further into the holding means 230. Upon sufficiently great actuation of the push button 1000 and thus correspondingly great displacement of the release element 210, the securing of the treatment tool 7 in the holding means 230 is released and the treatment tool 7 can be removed from the tool channel 240 of the headpiece 20. Such push-button mechanisms are usually implemented in a cover that is able to be screwed onto the headpiece 20 of the hand-held instrument 1.

This previous arrangement with the push button 1000 and the compression spring 1040 for releasing the holding mechanism requires a correspondingly high exertion of force, however, since, in addition to the spring force of the compression spring 1040 that is to be overcome, the interaction between the holding means 230 and release element 210 also requires a certain force—usually about 50 Newton. Thus, this previous mechanism is difficult to operate and impractical.

Furthermore, with this known mechanism, even slight actuation of the push button 1000 results in the pressure element 1001 contacting the release element 210, wherein, for this purpose, only the spring force of the compression spring 1040 needs to be overcome. However, contact of the push button 1000 with the release element 210 during operation represents a significant safety hazard, since, upon contact of the pressure element 1001 with the release element 210 rotating at high speed, considerable friction occurs, with the result that the push button 1000 becomes extremely hot extremely quickly, this in turn representing a significant risk of injury. Such slight pushing in can occur even through undesired contact of the push button 1000 with a tooth or a cheek in the mouth of a patient, with the result that there is in this case a risk of the patient being burnt. There is also a risk of the treating doctor being burnt, since it is quite conceivable for the latter to accidentally push the push button 1000 in slightly themselves during the treatment.

Such known instrument heads having a push-button mechanism for releasing the securing of a treatment tool are therefore difficult and impractical to operate and furthermore represent a hazard potential both for the treating doctor and for the patient.

Furthermore, EP 1 627 611 A1 describes a handpiece head for a medical handpiece, in which a stroke movement along an axis is transferred to a tool arranged in the handpiece head. In that case, the tool is able to be fixed in any desired position with respect to a rotation about the axis, wherein, depending on the requirement, the tool can rotate about its own axis or be prevented from such a rotation by a fixing mechanism.

For the fixing mechanism, the establishment of a clamping connection is provided. To that end, the handpiece head has a sleeve for receiving the tool, on which sleeve a first clamping part is arranged. Moreover, as part of an attachment on a grip part of the handpiece, a second clamping part is provided, which can be pushed in the direction of the handpiece head and in that case interacts with the first clamping part in such a way that the desired clamping connection is brought about. The clamping mechanism described therein thus serves primarily for locking the sleeve holding the tool in relation to the axis of rotation, but not for releasably securing the tool.

### SUMMARY OF THE INVENTION

The invention is based on the object of specifying a medical hand-held instrument having an improved mechanism for operating the release element and the holding means for releasing the securing of a treatment tool inserted into the hand-held instrument, and of specifying a cover having such an improved mechanism for a medical hand-held instrument.

This object is achieved according to the invention by the subject matter set out in the independent claims. Particular embodiments of the invention are specified in the dependent claims.

According to the invention, a medical, in particular dental hand-held instrument having a grip sleeve and a headpiece which is arranged at a front end of the grip sleeve and has a tool receiving opening is provided, wherein a holding means for rotatably securing a treatment tool (for example a dental drill) is arranged in the interior of the headpiece. The invention also provides that the holding means is arranged along an axis of rotation for the treatment tool, and that a release element is arranged in the interior of the headpiece along this axis of rotation, said release element being designed to interact with the holding means, wherein the release element releases the securing of the treatment tool upon interaction with the holding means. According to the invention, the headpiece furthermore has a cover, wherein the cover comprises a lever mechanism which has a first lever and a second lever which cooperate in order to release the securing of the treatment tool.

As a result, the mechanism for releasing the securing of the treatment tool is realized by means of a lever mechanism, wherein, in particular, the force to be exerted to release the securing is greatly minimized by the configuration of two cooperating levers.

In addition to the hand-held instrument according to the invention, a cover for a headpiece of a medical, in particular dental hand-held instrument is also provided according to the invention.

As a result, the cover is able to be attached in particular also to older medical hand-held instruments that are already known, such that the associated improved mechanism for releasing the holding mechanism is also usable with other and in particular older hand-held instruments. Thus, the advantages of the greater safety of use and easier usability of the cover according to the invention are also able to be experienced by users of older hand-held instruments.

The following embodiments and designs of the cover are therefore equally valid for the cover according to the invention and for the hand-held instrument according to the invention.

In one preferred design, the first lever, upon actuation, acts on a free end of the second lever. As a result, a particularly efficient lever action is achieved through the cooperation of the first and the second lever, such that the alleviation of the required force exertion for releasing the securing by means of the release element and holding means is increased.

Preferably, a pivot axis of the first lever and a pivot axis of the second lever are arranged parallel to one another and are perpendicular to a plane which extends along the axis of rotation. With such a design, a particularly space-saving arrangement and a particularly advantageous transmission of force from the first lever to the second lever can be achieved.

Preferably, the pivot axis of the first lever and the pivot axis of the second lever are arranged on the cover opposite one another in relation to the axis of rotation. As a result, a particularly advantageous arrangement of the two levers can be realized. Furthermore preferably, in the direction of the axis of rotation, the two pivot axes are offset from one another. With the additional offset arrangement, a correspondingly increased lever action can be realized, since the two pivot axes are positioned correspondingly at a spacing from one another and thus the potential lever length is increased.

Preferably, the second lever has a bend which, to interact with the release element, presses on the latter, wherein a force acting on the release element is limited by the bend. In this case, the invention provides in particular that the second lever, as a result of the bend, has elasticity, such that, from a particular applied lever force, the force acting on the release unit is limited and excessive force exertion is prevented. As a result, the safety of use of the hand-held instrument is increased since damage to components of the hand-held instrument is avoided.

Preferably, the second lever is provided, in the region of its pivot axis, with a return spring, in particular a leg spring, which is designed, in the absence of actuation of the first lever, to transfer the first and second levers into a rest position, wherein, in the rest position, the second lever is spaced apart from the release element. This has the simple effect that, following actuation of the lever mechanism—with the first and second levers—the two levers automatically return to their rest position, such that, in particular, contact of the second lever with the release element is prevented. Thus, the safety of use is further improved by this implementation.

Preferably, the first lever is provided, in its end region near the pivot axis, with lateral protrusions which serve for mounting the first lever in the cover and each engage laterally over the end region of the second lever. As a result of this configuration of the first lever, the second lever is mounted in a secured manner when the lever mechanism is not in use (rest position), wherein, furthermore, the position of the second lever with respect to the first lever is also fixed. These lateral protrusions of the first lever furthermore also increase the stability of the first lever.

Preferably, the first lever is provided, in its end region remote from the pivot axis, with a grip element. As a result, the operability of the lever mechanism for releasing the holding mechanism is simplified, wherein, furthermore, the transmission of force to the first lever is also improved. The grip element also additionally stabilizes the first lever.

Preferably, the first lever is provided, on its underside facing the second lever, with a recess which receives at least a part of the second lever in a rest position of the two levers. As a result, the second lever is prevented from shifting, wherein, in addition, the cover is able to be realized with a smaller overall height since the two levers are arranged in a space-saving manner.

Preferably, the cover is able to be coupled reversibly to the headpiece of the hand-held instrument, wherein preferably, for this purpose, the cover has a thread and the headpiece has a corresponding mating thread. As a result, maintenance, cleaning and repair work can be carried out in the interior of the head piece and on the cover particularly easily and advantageously, wherein, furthermore, the individual components of the cover and of the headpiece are particularly easily accessible in this way.

Preferably, the cover has a fixing device, wherein the lever mechanism is able to be locked selectively in a desired orientation with respect to the axis of rotation by means of the fixing device. As a result, the actuation direction of the lever mechanism can be put into an orientation preferred by the user, such that the lever mechanism is able to be personalized particularly easily and manageably.

In a specific preferable embodiment, the lever mechanism is mounted movably along the axis of rotation, and the lever mechanism is preloaded by means of spring force into a position spaced apart from the release element, wherein the lever mechanism is able to be depressed counter to the spring force in order, in the depressed state, to contact the release element in a rest position of the two levers. With this specific configuration, in addition to the lever mechanism with the first and second levers, an additional pushing mechanism for releasing the holding mechanism is implemented, wherein, for this purpose, the cover, together with the lever remaining in the rest position, is able to be depressed such that the second lever is contacted by the release element, with the result that, in turn, the securing in the holding means is released. In this sense, this embodiment provides two mechanisms for releasing the holding mechanism.

Preferably, the holding means is a collet, and/or the release element is a wedge element having a wedge-shaped head region and a planar bottom region. As a result, a particularly simple but robust holding mechanism made up of the release element and holding means can be realized.

Furthermore preferably, the first lever is in the form of a two-armed lever and the second lever is in the form of a one-armed lever. As a result, the lever action of the lever mechanism is enhanced, wherein, at the same time, the two levers are able to be positioned in a space-saving manner in the cover.

### BRIEF DESCRIPTION OF THE DRAWINGS

The invention is explained in more detail in the following text by way of an exemplary embodiment and with reference to the drawings, in which:

FIG. 1 shows a schematic side illustration of one embodiment of a medical hand-held instrument according to the invention with a treatment tool inserted into the headpiece of the hand-held instrument;

FIG. 2 shows a schematic cross-sectional illustration of a first exemplary embodiment of a cover and medical hand-held instrument according to the invention;

FIG. 3A shows a schematic cross-sectional illustration of a second exemplary embodiment of a cover according to the invention for a medical hand-held instrument with the lever mechanism in an operating position;

FIG. 3B shows a perspective illustration of the embodiment in FIG. 3A of a cover according to the invention for a medical hand-held instrument with the lever mechanism in an operation position;

FIG. 4A shows a schematic cross-sectional illustration of the embodiment in FIG. 3A of a cover according to the invention for a medical hand-held instrument with the lever mechanism in the rest position;

FIG. 4B shows a perspective illustration of the embodiment in FIG. 3A of a cover according to the invention for a medical hand-held instrument with the lever mechanism in the rest position;

FIG. 5 shows a schematic cross-sectional illustration of a third exemplary embodiment of a cover and medical hand-held instrument according to the invention;

FIG. 6 shows a schematic cross-sectional illustration of the head region of a medical hand-held instrument known from the prior art.

### DETAILED DESCRIPTION

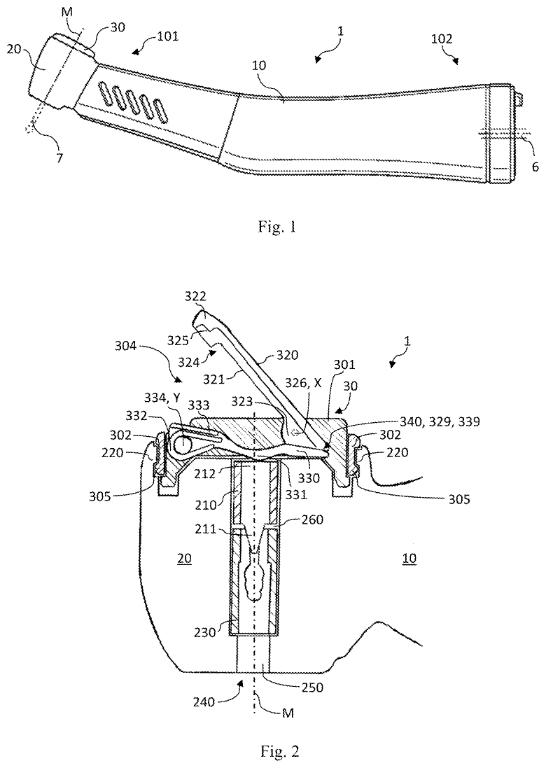

FIG. 1 shows the basic structure of a medical, and in particular dental, hand-held instrument 1 according to the invention, which has a headpiece 20 at its front end 101, wherein the headpiece 20 is in turn coupled to the grip sleeve 10 of the hand-held instrument 1. At the rear end 102 of the hand-held instrument 1, a connection for a supply line 6 is usually provided, via which the hand-held instrument 1 can be coupled for example to an energy source; in particular, provision can be made for the hand-held instrument 1 to be supplied with electrical energy or mechanical energy (for example rotational energy) via the latter.

The part of the hand-held instrument that is essential to the invention is illustrated in more detail in FIGS. 2 to 5, wherein the region of the headpiece 20 is illustrated in more detail here. The invention is aimed at an improved mechanism for releasing the holding mechanism for a treatment tool 7 in the headpiece 20 of the hand-held instrument 1. This mechanism is in this case installed in a cover 30 of the hand-held instrument 1 and is embodied as a lever mechanism 304. The cover 30 is always able to be reversibly coupled to the headpiece 20 in the various exemplary embodiments shown here.

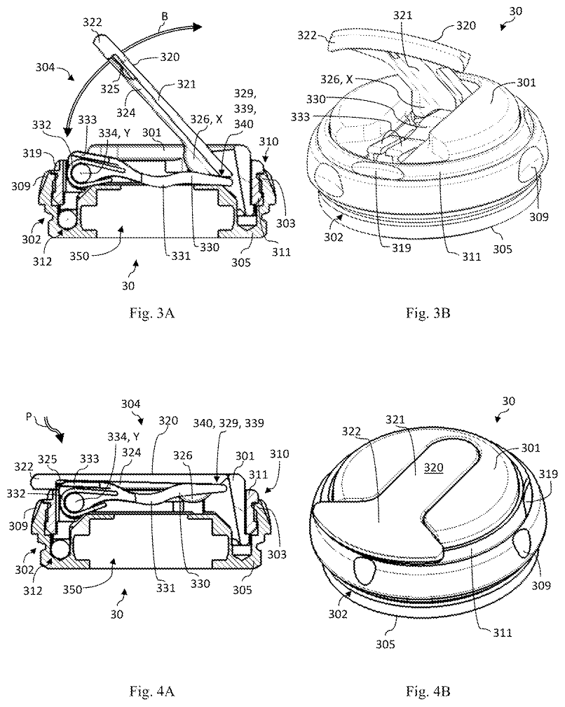

The lever mechanism 304 has a first lever 320 and a second lever 330, which cooperate with one another in order to release the securing of the treatment tool 7. To this end, the lever mechanism 304 interacts with the release element 210 such that the release element 210 in turn actuates (or opens) the holding means 230 and a corresponding treatment tool 7 can be inserted into or removed from the headpiece 20 of the hand-held instrument 1. As a result of the lever mechanism 304, the force to be exerted by the user is minimized. Furthermore, the actuation B of the first lever 320 takes place in the opposite direction to the interaction of the second lever 330 with the release element 210, such that, as a result of this necessary actuation movement B, spreading away from the cover 30, of the first lever 320, as is illustrated by way of example in FIG. 3A, unintentional triggering of the lever mechanism 304 for releasing the holding mechanism is prevented. As a result, there is no risk of the user and the patient being burnt by unintentional actuation, since force exertion on the cover 30 and in particular the lever 320—for example by contact with a cheek or a tooth of a patient, or a finger of the treating doctor—does not trigger the lever mechanism 304, and so, during operation of the hand-held instrument 1, contact of the lever mechanism 304 with rapidly rotating components is avoided.

Independently of the specific embodiment of the cover 30, the lever mechanism 304 implemented therein is always designed, upon actuation B, to interact with the release element 210 such that the latter in turn releases the holding means 230. The holding means 230 and the release element 230 in this case form a so-called holding mechanism. As is also illustrated in FIG. 2, the holding means 230 is preferably a collet—i.e. a tubular sleeve which has a stepped bore and at least one slot. The holding means 230 is, upon actuation, spread open by means of the release element 210, which preferably has a wedge-shaped head region 211 and a planar bottom region 212, wherein, for this purpose, for example the wedge-shaped head region 211 of the release element 210 interacts with the slot of the collet and thus expands the sleeve of the collet such that the treatment tool 7 can be inserted into or removed from the holding means 230 via the tool receiving opening 240 and the tool channel 250 of the headpiece 20. For this purpose, the release element 210 has to be pushed actively in the direction of the holding means 230—this being achieved in particular by actuation B of the lever mechanism 304—since the interaction of the two components of the holding mechanism is self-locking, i.e. the release element 210 is pushed out by the holding means 230 itself.

Within the head part 20, the release element 210 and the holding means 230 are arranged in a recess 260 provided for this purpose. The diameter of this recess 260 is in this case greater than the diameter of the tool receiving opening 240, and, respectively, of the tool channel 250, such that the components of the holding mechanism are mounted securely in the interior of the headpiece 20. In particular, provision is made for both the holding means 230 and the release element 210 to be mounted so as to be rotatable together with the treatment tool 7, since they corotate at the speed of the treatment tool 7 during use of the hand-held instrument 1. In this case, the components of the holding mechanism additionally assume a tool-centring function.

FIG. 2 shows a first exemplary embodiment of the cover 30. The cover 30 is in this case screwed together, via a thread 302, with the headpiece 20, which has a corresponding mating thread 220. The lever mechanism 304 is placed in the central piece 301 of the cover 30, wherein, in this first exemplary embodiment, the central piece 301 is arranged in a freely rotatable manner relative to the outer piece 305 annularly surrounding central piece 301, such that, depending on the situation, the orientation of the lever mechanism 304 and in particular of the first lever 320 operated by the user can be changed. The thread 302 is arranged on the outer piece 305.

The first lever 320 (also referred to as upper lever 320) has a grip element 322, which makes it much easier to operate the lever mechanism 304. On the opposite side from the grip element 322, the first lever 320 is provided with lateral protrusions 323, which each have an attachment 326 which serve for mounting the lever 320 in the cover 30 and each engage laterally over the end region of the second lever 330. As a result, the two levers 320, 330 are arranged in the cover 30 in a space-saving manner in the rest position P, as indicated in FIG. 4A. The lateral protrusions 323 form, with their attachments 326, the pivot axis X of the first lever 320, which is arranged on the first lever 320 such that this lever is a two-armed lever. The pivot axis X of the first lever 320 is in this respect spaced apart both from the grip element 322 and from the opposite end of the first lever 320 from the grip element 322.

The opposite end of the first lever 320 from the grip element 322 forms the contact face 329 of the first lever 320, said contact face, upon actuation B of the lever mechanism 304, acting on a free end of the second lever 330. This free end of the second lever 330 has the contact face 339, which interacts with the contact face 329 of the first lever 320. The grip element 322 and the contact face 329 of the first lever 320 are connected via an elongate bar 321. The contact 340 between the first lever 320 and second lever 330 takes place via the contact faces 329, 339, always exists and is not interrupted even in the rest position P of the two levers 320, 330, such that even slight actuation B of the first lever 320 results in a movement of the second lever 330.

Furthermore, the first lever 320 is provided, on its underside facing the second lever 330, with a recess 324 which, in a rest position P of the two levers 320, 330, receives at least a part of the second lever 330 such that the second lever 330 can be embedded therein in a space-saving manner.

The second lever 330 (also referred to as lower lever 330) is embodied as a one-armed lever, wherein the attachment 334 thereof is arranged at the opposite end of the second lever 330 from the contact face 339 of the second lever 330. This attachment 334 of the second lever 330 additionally forms the pivot axis Y thereof.

Between the contact face 339 and the attachment 334, the second lever has a bend 331, which, in order to interact with the release element 210, presses on the latter, wherein a force acting on the release element 210 is limited by the bend 331. As a result of this bend 331, the second lever 330 has elasticity, such that, from a particular lever force, the lower lever 330 bends no further and thus excessive force exertion on the release element 210 is prevented. The bend 331 is in this case arranged centrally in the cover 30 such that, independently of the orientation of the central piece 301, it is always placed on the axis of rotation M, and thus, upon actuation B, interacts with the release element 210 and ensures optimal transmission of force.

The second lever 330 is furthermore provided, in the region of its pivot axis Y, with a return spring 332, in particular a leg spring, which is designed, in the absence of actuation B of the first lever 320, to transfer the first and second levers 320, 330 into a rest position P. The return spring 332 in this case has a flat support 333, against which the return spring 332 acts, resulting in a restoring force which pushes the second lever upwards—i.e. in the direction of the first lever 320—, with the result that the upper lever 320 in turn snaps back and is pushed in the direction of the rest position P, as long as the first lever 320 is not actuated.

The attachment 326 of the first lever 320 and the attachment 336 of the second lever 330, or the pivot axes X, Y thereof, are arranged parallel to one another and are also perpendicular to a plane which extends along the axis of rotation M. This results in a particularly advantageous transmission of force from the lever mechanism 304 to the release element 210 positioned on the axis of rotation M.

Furthermore, the attachment 326 of the first lever 320 and the attachment 336 of the second lever 330, or the pivot axes X, Y thereof, are arranged on the cover opposite one another in relation to the axis of rotation M, wherein, as a result of the two-armed configuration of the first lever 320 and the one-armed configuration of the second lever 330, particularly efficient transmission of force is allowed. As a result of the first and second lever 320, 330 being arranged one on top of the other, the two pivot axes X, Y are furthermore also offset slightly from one another in the direction of the axis of rotation M.

In the rest position P, the second lever 330 is spaced apart from the release element 210, such that unintentional contact between these two elements is precluded.

Furthermore, the first lever 320 has preferably on the underside of the grip element 322 a latching element 325, which, in the rest position P, interacts and latches together with the second lever 330. As a result, the user is provided with haptic and acoustic feedback, as long as the two levers 320, 330 are correctly in the rest position P. This also increases the operational safety of the hand-held instrument 1, since, as a result of the latching, additional mechanical securing of the lever mechanism 304 is formed, and so unintentional lifting of the first lever 320 and thus actuation B of the lever mechanism 304 is prevented.

As a result of the two-part configuration of the lever mechanism 304, the force to be exerted by the user is considerably reduced. Although, with a one-part lever mechanism, alleviation would also be perceptible, upon actuation, the one lever would then necessarily rub against the release element 210, since the contact face of the one lever then shifts laterally on the release element 210, this resulting in disadvantageous material abrasion on both elements. This would considerably reduce the operational safety of the hand-held instrument 1. In this regard, the two-part lever mechanism 304 proves to be advantageous for the present application.

FIGS. 3A, 3B, 4A and 4B show a second exemplary embodiment of a cover 30 according to the invention, which is able to be reversibly coupled to a hand-held instrument 1. The structure of this second embodiment is substantially identical to the first exemplary embodiment already known from FIG. 2, wherein the illustration without the other components of the hand-held instrument 1 and thus in particular also without the illustration of the components of the holding mechanism make the spatial structure of the cover 30 more clearly apparent. In this regard, FIGS. 3B and 4B show a perspective view of the cover 30 and the lever mechanism 304 thereof, which is identical to the first embodiment.

The second embodiment involves a further additional feature which additionally increases the operability and operational safety. In this case, an additional fixing device 310 is provided, which makes it possible to lock a set orientation with respect to the axis of rotation M of the lever mechanism 304 such that, following locking, unintentional turning of the central piece 301 is prevented. Of course, the locking of the fixing device 310 can be released again such that the lever mechanism 304 and, associated therewith, also the central piece 301 can be reoriented in order then to be locked again. As is apparent from FIGS. 3A to 4B, the fixing device 310 is, in the variant illustrated here, an over-nut device, consisting of a ring element 311, which in turn has a thread and is designed to interact with an internal thread 303 arranged on the inner side of the threaded piece. The ring element 311 is in this case arranged between the central piece 301 and outer piece 305, and, as long as the ring element 311 has been screwed in appropriately, fixes the central piece 301 in position by way of a force fit. For this purpose, the ring element 311 has, for screwing in, corresponding tool indentations 319, such that secure locking is allowed. As a result of this embodiment with the fixing device 310, the hand-held instrument 1, or the cover 30, can be adapted to the individual requirements and preferences of the user, such that, for example, a left-handed person can orient the lever mechanism 304 differently, thereby substantially increasing the ergonomics of the hand-held instrument 1.

Furthermore, the outer piece 305 can also have corresponding tool indentations 309, thereby making it possible to firmly screw the cover 30 together with the headpiece 20 of the hand-held instrument 1, in particular in such a way that it is not possible to uncouple these components without a tool, this further increasing the operational safety of the hand-held instrument 1.

As is also apparent from FIGS. 3A and 4B, the outer piece 305 can also extend beneath the central piece 301, this increasing the stability of the cover. In this case, provision can furthermore be made for the central piece 301 to be rotatably mounted in the outer piece 305 via a ball bearing 312, such that reorientation of the central piece 301 and thus also of the lever mechanism 304 along the axis of rotation M is simplified. By means of the fixing device 310, the ball bearing 312 is then likewise locked, such that further turning of the central piece 301 is prevented. These configurations are likewise applicable, as a whole or individually, to the first embodiment previously shown in FIG. 2.

The cover 30 furthermore also has a receiving space 350, in which components of the headpiece 20 are received when the cover 30 is coupled to the hand-held instrument 1. It is important here that the freedom of movement of the second lever 330 is not restricted, such that the bend 331 can interact with the release element 210 of the headpiece 20 upon actuation B of the lever mechanism 304. This is also transferable to the embodiment previously shown in FIG. 2.

FIGS. 3A and 3B furthermore show actuation positions B of the lever mechanism 304, wherein, viewed together with FIGS. 4A and 4B, which show the rest position P of the lever mechanism 304, the possible lever positions are demonstrated. These illustrations of the actuation B and of the rest position are in this case transferable to all exemplary embodiments, wherein, of course, differently arranged two-part lever mechanisms are also conceivable.

As is apparent in particular from FIGS. 4A and 4B, the lever mechanism 304 is formed such that the first lever 320 is integrated in a planar manner into the surface of the central piece 301. Only the grip element 322 protrudes somewhat from the central piece 301 such that it can be used particularly easily by a user.

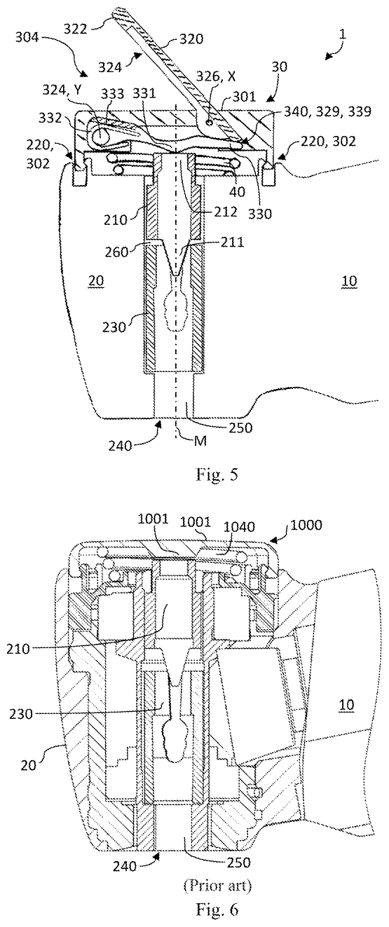

FIG. 5 illustrates a further, third exemplary embodiment of the cover 30 and of the hand-held instrument 1. In addition to the two-part lever mechanism 304 known from the first and second embodiments, this embodiment has a further mechanism for releasing the holding mechanism.

In this case, a spring element 40 is arranged between the cover 30 and headpiece 20. In this embodiment, the cover 30 and thus also the lever mechanism 304 are mounted movably along the axis of rotation M, wherein the spring element 40 pushes the cover 30 away from the headpiece 20. In this regard, the lever mechanism 304 is preloaded by means of the spring force of the spring element 40 into a position spaced apart from the release element 210.

The cover 30 is coupled to the headpiece 20 in this case likewise via the thread 302 and the mating thread 220, but this coupling is now embodied such that, after the cover 30 has been screwed on via the threads 302, 220, the cover 30 can be displaced back and forth along a smooth surface, wherein the threads 302, 220 prevent unintentional uncoupling of the cover 30 from the headpiece 20. In this embodiment, the lever mechanism 304 is thus able to be depressed counter to the spring force, in order, in the depressed state, to contact the release element 210 in a rest position of the two levers 320, 330.

In this regard, with this third exemplary embodiment, both the lever mechanism 304 and a push-button mechanism for releasing the holding mechanism are integrated in the cover 30, such that a user has the choice of which mechanism to use. In this embodiment, provision can be made, for example, to use a particularly strong spring element 40 which is not compressed by light contact or exertions of force on the cover 30, such that unintentional contacting of the lever mechanism 304 during operation of the hand-held instrument 1 is prevented. In this case, however, the user of the hand-held instrument 1 would need to use great physical effort to couple the treatment tool 7 to the hand-held instrument 1 (i.e. to clamp it) or to uncouple it therefrom (i.e. to release it). Users who cannot exert this necessary force can then use the lever mechanism 304 in order to easily release the holding mechanism of the hand-held instrument 1.

All of the above-described exemplary embodiments of the hand-held instrument 1 and of the cover 30 make it much easier to release the treatment tool 7. The cover 30 is in this case advantageously designed such that it is likewise able to be screwed onto older hand-held instruments 1 that are already known, such that the two-part lever mechanism 304 according to the invention can also be used on older hand-held instruments 1. With the design of a removable cover 30, maintenance, cleaning and repair work can be carried out in the interior of the headpiece 20 and on the cover 30 itself particularly easily and advantageously. This allows a particularly long service life of the hand-held instrument 1 and of the cover 30.

The hand-held instrument 1 designed in this way, and, respectively, the cover 30, designed in this way, for a hand-held instrument 1, thus allow particularly easy and safe operation for releasing the holding mechanism of a hand-held instrument 1 for a treatment tool 7. Unintentional actuations of the mechanism are thus avoided, wherein contact of the lever mechanism 304 with the release element 210, in particular during operation, is also prevented, and so there is no risk of the patient or the treating doctor being burnt.

1. Medical, in particular dental hand-held instrument having a grip sleeve and a headpiece which is arranged at a front end of the grip sleeve and has a tool receiving opening,
wherein a holding means for rotatably securing a treatment tool, for example a dental drill, is arranged in the interior of the headpiece,
wherein the holding means is arranged along an axis of rotation for the treatment tool,
wherein a release element is arranged in the interior of the headpiece along the axis of rotation, said release element being designed to interact with the holding means, wherein the release element releases the securing of the treatment tool upon interaction with the holding means, and
wherein the headpiece furthermore has a cover, wherein the cover comprises a lever mechanism which has a first lever and a second lever which cooperate in order to release the securing of the treatment tool,
characterized in that the first lever, upon actuation, acts on a free end of the second lever.

2. (canceled)

3. Medical hand-held instrument according to claim 1, characterized in that a pivot axis of the first lever and a pivot axis of the second lever are arranged parallel to one another and are perpendicular to a plane which extends along the axis of rotation.

4. Medical hand-held instrument according to claim 3, characterized in that the pivot axis of the first lever and the pivot axis of the second lever are arranged on the cover opposite one another in relation to the axis of rotation,
wherein, in the direction of the axis of rotation, the two pivot axes are preferably offset from one another.

5. Medical hand-held instrument according to claim 1, characterized in that the second lever has a bend which, to interact with the release element, presses on the latter, wherein a force acting on the release element is limited by the bend.

6. Medical hand-held instrument according to claim 1, characterized in that the second lever is provided, in the region of its pivot axis, with a return spring, in particular a leg spring, which is designed, in the absence of actuation of the first lever, to transfer the first and second levers into a rest position, wherein, in the rest position, the second lever is spaced apart from the release element.

7. Medical hand-held instrument according to claim 1, characterized in that the first lever is provided, in its end region near the pivot axis, with lateral protrusions which serve for mounting the lever in the cover and each engage laterally over the end region of the second lever.

8. Medical hand-held instrument according to claim 1, characterized in that the first lever is provided, in its end region remote from the pivot axis, with a grip element.

9. Medical hand-held instrument according to claim 1, characterized in that the first lever is provided, on its underside facing the second lever, with a recess which receives at least a part of the second lever in a rest position of the two levers.

10. Medical hand-held instrument according to claim 1, characterized in that the cover is able to be coupled reversibly to the headpiece of the hand-held instrument, wherein preferably, for this purpose, the cover has a thread and the headpiece has a corresponding mating thread.

11. Medical hand-held instrument according to claim 1, characterized in that the cover has a fixing device, wherein the lever mechanism is able to be locked selectively in a desired orientation with respect to the axis of rotation by means of the fixing device.

12. Medical hand-held instrument according to claim 1, characterized in that the lever mechanism is mounted movably along the axis of rotation, and in that the lever mechanism is preloaded by means of spring force into a position spaced apart from the release element, wherein the lever mechanism is able to be depressed counter to the spring force in order, in the depressed state, to contact the release element in a rest position of the two levers.

13. Medical hand-held instrument according to claim 1, characterized in that the holding means is a collet, and/or the release element is a wedge element having a wedge-shaped head region and a planar bottom region.

14. Cover for a headpiece of a medical, in particular dental hand-held instrument having a grip sleeve and a headpiece which is arranged at a front end of the grip sleeve and has a tool receiving opening,
wherein a holding means for rotatably securing a treatment tool, for example a dental drill, is arranged in the interior of the headpiece,
wherein the holding means is arranged along an axis of rotation for the treatment tool,
wherein a release element is arranged in the interior of the headpiece along the axis of rotation, said release element being designed to interact with the holding means, wherein the release element releases the securing of the treatment tool upon interaction with the holding means, and
wherein the cover comprises a lever mechanism which has a first lever and a second lever which cooperate in order to release the securing of the treatment tool;
characterized in that the first lever, upon actuation, acts on a free end of the second lever.

15. Cover for a headpiece according to claim 14,
characterized in that the first lever is in the form of a two-armed lever and the second lever is in the form of a one-armed lever.
